(19) United States
(12) Patent Application Publication
(10) Pub. No.: US 2018/0353710 A1
(43) Pub. Date: Dec. 13, 2018

(54) PROTECTIVE SLEEVE FOR SYRINGE

(71) Applicant: Wing Young Co., Ltd., Taoyuan (TW)

(72) Inventors: Mao-Pang CHEN, Taoyuan City (TW); Wei-Lun CHEN, Taoyuan City (TW)

(21) Appl. No.: 15/916,709

(22) Filed: Mar. 9, 2018

(30) Foreign Application Priority Data
Jun. 9, 2017 (TW) 106119278

(51) Int. Cl. A61M 5/32

(52) U.S. Cl. CPC A61M 5/3245; A61M 5/3129; A61M 5/3202; A61M 5/3271

(57) ABSTRACT

The present disclosure illustrates a protective sleeve for a syringe. The protective sleeve includes a hollow pipe body and an outer sleeve. The pipe body includes a channel passing therethrough axially, and a connection part formed at a side thereof and configured to combine with the preset syringe. The outer sleeve is movably and extendably mounted on the pipe body. A hollow body of the outer sleeve includes a through hole passing through axially and configured for insertion of the pipe body, and a covering part formed at an end thereof. After the syringe is injected already, the outer sleeve can be extended outwardly to make the covering part completely cover a needle of the syringe, thereby preventing the needle from being exposed out of the outer sleeve to stab person. As a result, the protective sleeve of the present can improve safety in using syringe.

This application claims the priority benefit of Taiwan patent application number 106119278, filed on Jun. 9, 2017.

### BACKGROUND OF THE INVENTION

### 1. Field of the Invention

The present disclosure relates to a protective sleeve for a syringe, more particularly to a protective sleeve which includes a pipe body configured to assemble on the syringe and an outer sleeve movably and extendably mounted on the pipe body, and when the outer sleeve is moved to extend outwardly, a covering part of the outer sleeve can completely cover a needle of the syringe, so as to prevent the needle from stabbing person to cause dangerous situation, and further prevent from occurrence of infection, thereby improving safety in using the syringe.

### 2. Description of the Related Art

Nowadays, in medical science technology field, the most common disposable syringe mainly comprises a cylinder, a putter and a needle. When using the disposable syringe for medical injection, the user can push the putter of the disposable syringe to push medicinal liquid stored inside the cylinder to flow through the needle which is inserted into the end of the cylinder, so as to complete injection. However, after the needle contacts the sick body such as a body part of patient, the needle is exposed out of the syringe; furthermore, after the syringe is injected already, health care worker must manually mount needle sleeve on the needle and then collect the needles for recycling, so as to prevent medical workers, health workers or waste workers from being stabbed by needles to be infected by blood-borne diseases, such as AIDS, hepatitis B, hepatitis C, tuberculosis, diphtheria, or typhoid fever.

However, if the medical worker is collided by an external force, or is distracted, or carelessly misaligns the needle with the needle sleeve during the process of mounting the needle sleeve over the needle, it is highly likely that the medical worker may accidentally be stabbed by the needle and infected by blood-borne disease. For this reason, patients, doctors, nurses, nursing staff and health workers, and all those who work in contact with the syringe, are in potential danger.

Therefore, what is needed is to develop a protective sleeve for syringe, to solve above-mentioned problems.

### SUMMARY OF THE INVENTION

In order to solve above-mentioned problems, the inventor develops a protective sleeve for syringe, based on collected data, years of experience, and multiple tests and modifications.

An objective of the present disclosure is to provide a protective sleeve for a syringe. The protective sleeve includes a pipe body, which includes a channel passing therethrough axially and a connection part disposed at a side thereof, and an outer sleeve movably and extendably mounted on the pipe body. A hollow body of the outer sleeve includes a through hole passing through axially and configured for insertion of the pipe body, and a covering part formed at an end thereof. The connection part of the pipe body is configured to assemble with the syringe. After the syringe is injected already, the outer sleeve can be moved to extend outwardly to make the covering part of the outer sleeve completely cover a needle of the syringe, so as to indeed prevent the needle from being exposed out of the outer sleeve, and further prevent the needle from stabbing person to cause dangerous situation, thereby improving safety in using the syringe. When moving the outer sleeve to extend outwardly, the injector can completely cover the needle of the syringe without passing a hand thereof in front of the tip of the needle of the syringe, so as to improve the safety in operating the syringe.

An objective of the present disclosure is that the connection part of the pipe body can be stably assembled with the syringe, so that the outer sleeve can be used without shaking or falling, thereby ensuring the stability in using the protective sleeve; furthermore, the protective sleeve can be assembled with the syringe by the connection part of the pipe body so that the protective sleeve can be applicable to most types of syringes in market, and it is not necessary to change the structural design of the syringe, thereby improving convenience in using the protective sleeve.

An objective of the present disclosure is that the pipe body is made of transparent material and the outer sleeve includes at least one hollow part cut therethrough, so as to facilitate the injector to observe, through the hollow part, the injection condition of the medicinal liquid stored in the cylinder of the syringe and further accurately inject the medicinal liquid; furthermore, a surface of the outer sleeve can be marked with text message or image message of instructions for use, warning instructions or slogans, so that the protective sleeve can be applied for multiple purposes.

An objective of the present disclosure is that a hole diameter of the covering part of the outer sleeve is smaller than a hole diameter of the hollow body of the outer sleeve, so as to prevent the injector's finger from contacting the tip of the needle of the syringe through opening of the covering part, thereby achieving the objective of indeed preventing infection during the recycling process.

### BRIEF DESCRIPTION OF THE DRAWINGS

The structure, operating principle and effects of the present disclosure will be described in detail by way of various embodiments which are illustrated in the accompanying drawings.

FIG. 1 is an elevational view of a protective sleeve of the present disclosure.

FIG. 2 is an exploded view of the protective sleeve of the present disclosure.

FIG. 3 is an elevational view of the protective sleeve of the present disclosure, before use.

FIG. 4 is an elevational view of the protective sleeve of the present disclosure, after use.

### DETAILED DESCRIPTION OF THE PREFERRED EMBODIMENTS

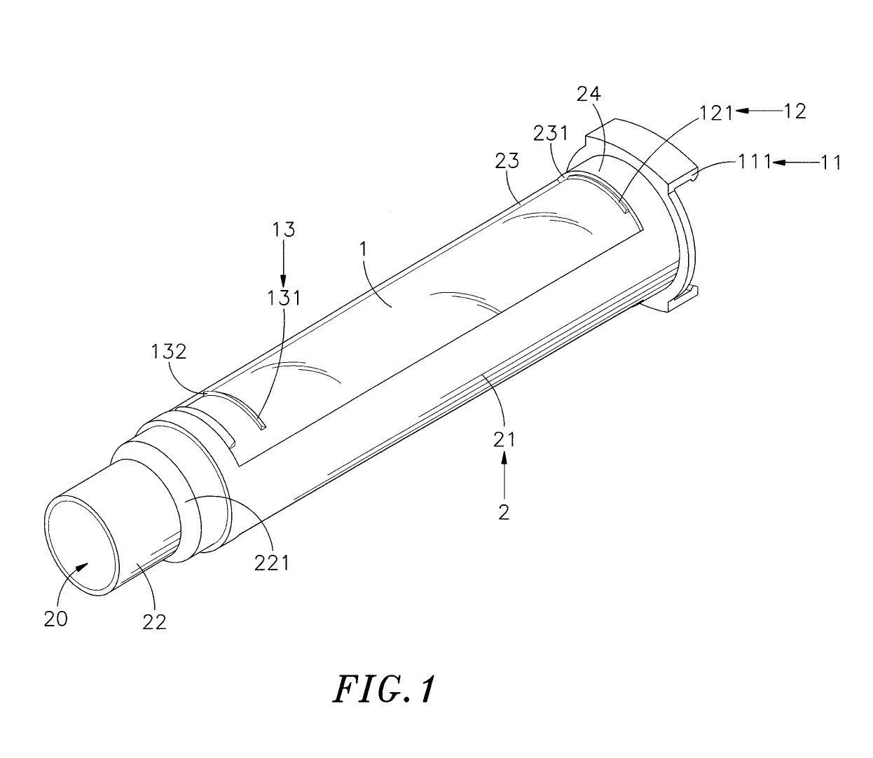

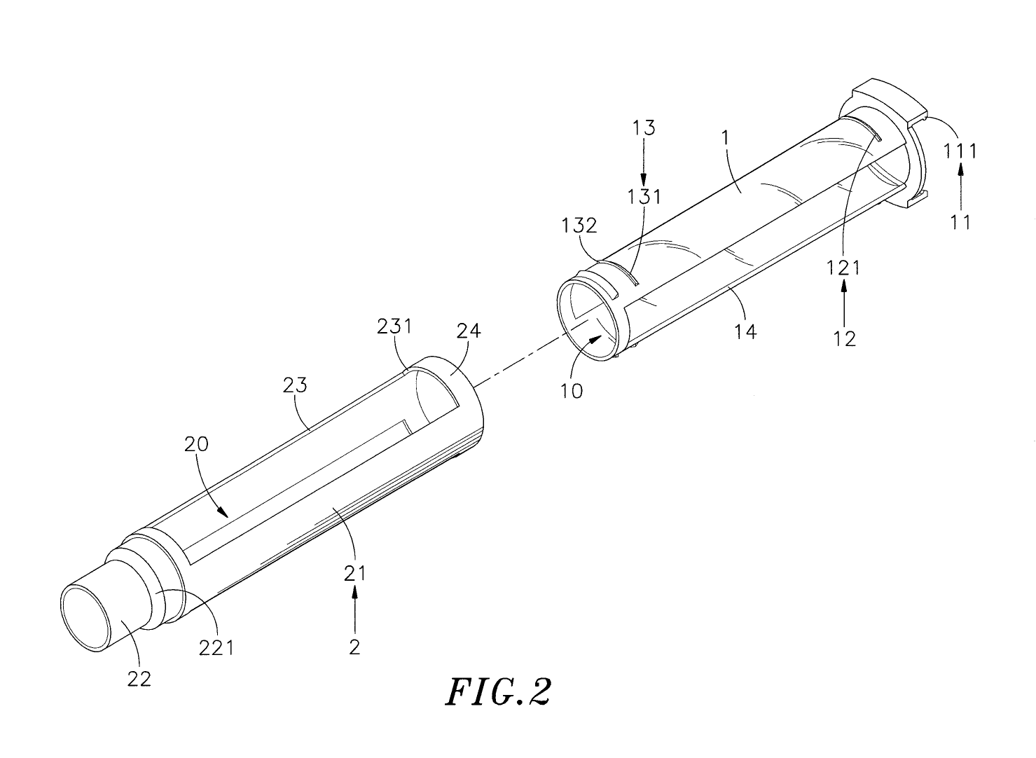

Please refer to FIGS. 1 and 2, which show elevational view and exploded view of the protective sleeve of the present disclosure. As shown in FIGS. 1 and 2, the protective sleeve of the present disclosure includes a pipe body 1 and an outer sleeve 2.

The pipe body 1 is made of transparent material, and is in a hollow shape and provided with a channel 10 passing therethrough axially. The pipe body 1 also includes a connection part 11 disposed at a side thereof. Two opposite sides of the connection part 11 are outwardly bent to form two clamping parts 111 in hook shape. The pipe body 1 also includes at least one position-limiting block 12 protruded on a surface of an end thereof and spaced apart from the connection part 11 by a distance, and each position-limiting block 12 includes an arc-shaped guiding surface 121 formed on a surface thereof. The pipe body 1 comprises stop blocks 13 protruded on a surface of other end thereof and axially arranged in interval. The two stop blocks 13 of the pipe body 1 comprise guiding inclines 131 separately formed on outer side surfaces thereof opposite to each other, and stop surfaces 132 separately formed on inner side surfaces thereof opposite to each other. The pipe body 1 comprises a cut hole 14 cut through the surface thereof.

The outer sleeve 2 includes a hollow body 21 which is provided with a through hole 20 passing therethrough axially. The hollow body 21 also includes a covering part 22 having gradually-reduced hole diameter and formed at an end thereof. The covering part 22 includes a tapered part 221 formed at a side thereof adjacent to the hollow body 21. The outer sleeve 2 comprises at least one hollow part 23 cut therethrough, and a position-limiting part 24 formed between the hollow part 23 and other end thereof. The hollow part 23 of the outer sleeve 2 comprises a supporting surface 231 formed on a side surface thereof adjacent to the position-limiting part 24.

In order to assemble aforementioned components, the pipe body 1 can be inserted into the through hole 20 of the outer sleeve 2, and the end of the hollow body 21 of the outer sleeve 2 away from the covering part 22 is passed through the stop blocks 13 and the position-limiting block 12 of the pipe body 1 in sequential order, so that the position-limiting part 24 of the outer sleeve 2 can be moved to a position between the position-limiting block 12 and the connection part 11 and be limited between the position-limiting block 12 and the connection part 11. During the process of passing the hollow body 21 through the stop blocks 13 and the position-limiting block 12 of the pipe body 1, the hollow body 21 of the outer sleeve 2 can be smoothly mounted on the outside of the pipe body 1 under the guiding effect of the guiding inclines 131 of the stop blocks 13 and the guiding surface 121 of the position-limiting block 12, so as to complete the assembly of the protective sleeve of the present disclosure.

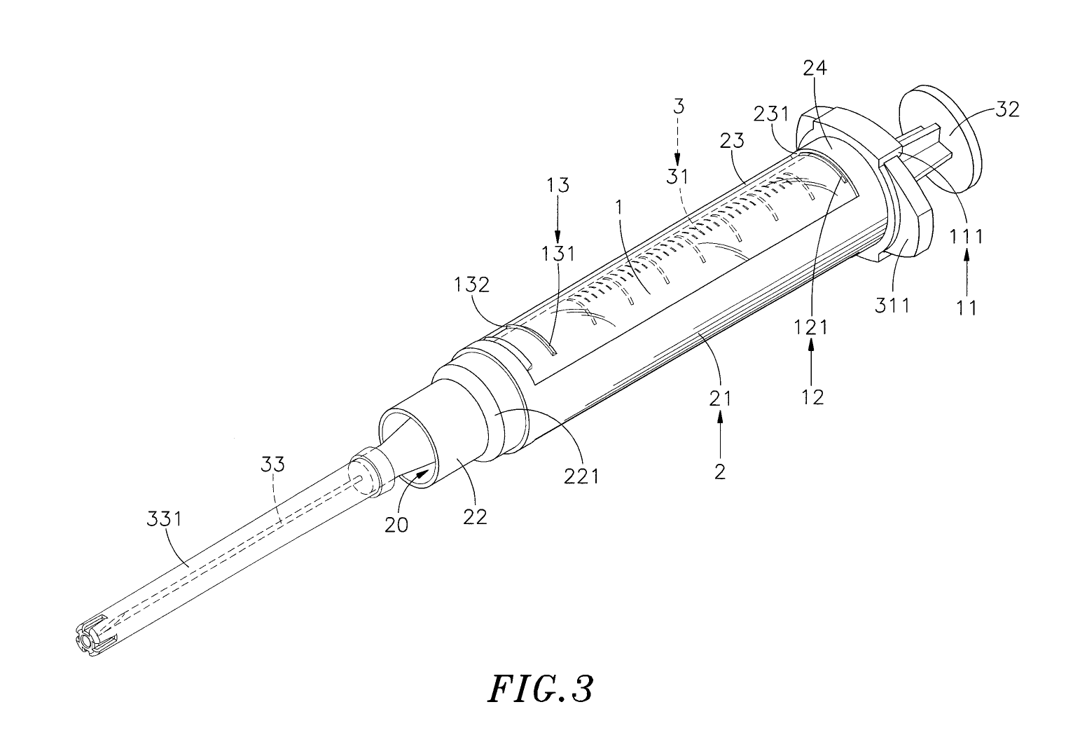

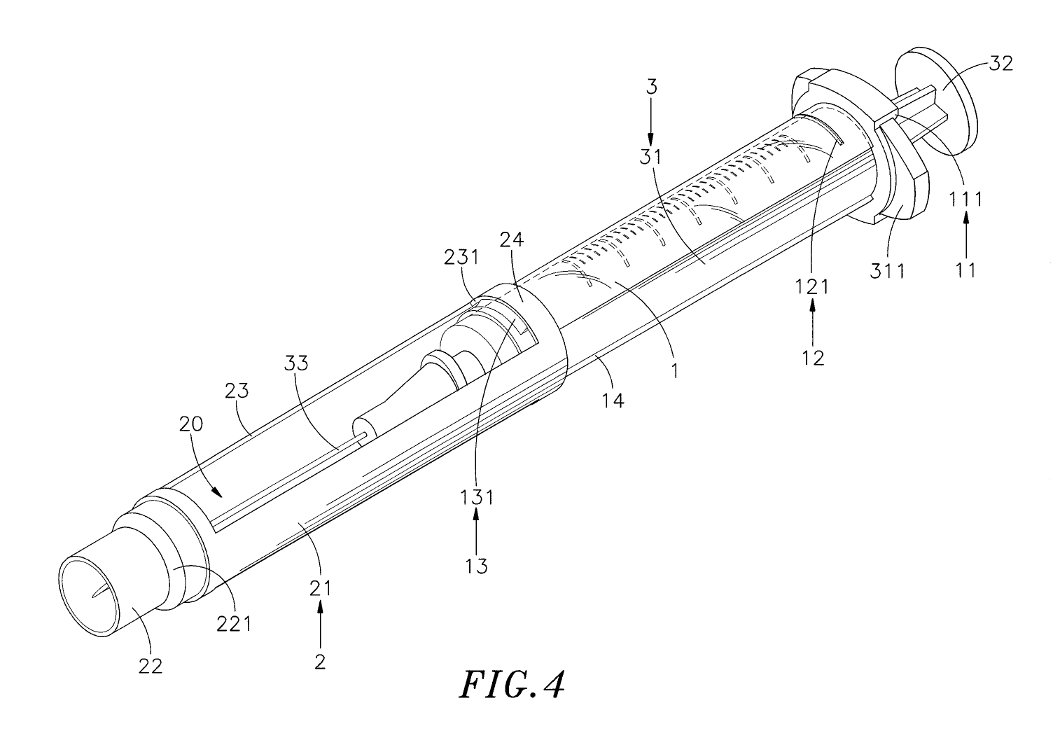

Please refer to FIGS. 3 and 4, which show elevational views of the protective sleeve of the present disclosure before use and after use, respectively. In practical use, the connection part 11 of the pipe body 1 can be used to combine with a syringe 3. The syringe 3 includes a cylinder 31, the cylinder 31 including a push plate 311 in an elliptical shape and outwardly formed at an end thereof, and a putter 32 inserted and reciprocated in the cylinder 31, and a needle 33 mounted on other end of the cylinder 31. A protective case 331 is mounted over the needle 33. In order to assemble the syringe 3 with the protective sleeve, the needle 33 can be inserted into, though, and out of the channel 10 of the pipe body 1, and then the pipe body 1 can be clamped and stably positioned with the periphery of the push plate 311 of the cylinder 31 by the clamping parts 111 of the connection part 11, so that the syringe 3 can be integrally assembled with the protective sleeve of the present disclosure stably.

When using the syringe 3, the injector can separate the protective case 331 from the needle 33, and add medicinal liquid (not shown in figures) into the cylinder 31 of the syringe 3, and then use the needle 33 to perform injection on the sick body, such as a body part of patient. The pipe body 1 is made of transparent material and the outer sleeve 2 is provided with at least one hollow part 23 cut therethrough, so that the injector can observe, through the hollow part 23, the injection condition of the medicinal liquid in the cylinder 31, to accurately inject the medicinal liquid. After the injection is completed, the injector can apply force to extend the outer sleeve 2 outwardly to release the position-limiting part 24 of the outer sleeve 2 from being limited by the position-limiting block 12 and the connection part 11, so that the outer sleeve 2 becomes movable and extendable on the surface of the pipe body 1. Next, the injector can continuously extend the outer sleeve 2 outwardly, the position-limiting part 24 of the outer sleeve 2 is then moved along the guiding incline 131 of the stop block 13 of the pipe body 1 to enter a position between the stop blocks 13, so that the position of the position-limiting part 24 is limited by the stop blocks 13. The two stop blocks 13 are provided with the stop surfaces 132 respectively formed on inner side surfaces thereof opposite to each other, so the supporting surface 231 of the hollow part 23 and an outer side surface of the position-limiting part 24 of the outer sleeve 2 are stopped and limited by the two stop surfaces 132, thereby preventing the position-limiting part 24 of the outer sleeve 2 from easily escaping from the position between the stop blocks 13 of the pipe body 1, to further provide nice positioning effect. As a result, the outer sleeve 2 can be prevented from being separated from the pipe body 1 when the outer sleeve 2 is extended outwardly. After the position-limiting part 24 of the outer sleeve 2 is positioned and limited between the stop blocks 13, the covering part 22 of the outer sleeve 2 can completely cover the needle 33 of the syringe 3, so as to effectively prevent the injector or other person, such as health worker or waste worker, from accidentally contacting the needle 33, and further prevent from occurrence of infection. As a result, the operation of the protective sleeve of the present disclosure is completed.

The protective sleeve of the present disclosure has following advantages.

First, the outer sleeve 2 is movable and extendable on the surface of the pipe body 1, so the injector can extend the outer sleeve 2 outwardly after the syringe 3 is injected already, to make the covering part 22 of the outer sleeve 2 completely cover the needle 33 of the syringe 3, thereby indeed preventing the needle 33 from being exposed out of the outer sleeve 2, and further preventing the needle 33 from stabbing person to cause dangerous situation, to improve safety in using the syringe 3; furthermore, while the outer sleeve 2 is moved to extend outwardly, the injector can completely cover the needle 33 of the syringe 3 without passing a hand thereof in front of the tip of the needle 33 of the syringe 3, so that the safety in using the protective sleeve can be improved.

Secondly, the protective sleeve can be stably assembled on the syringe 3 by the connection part 11 of the pipe body 1, so that the outer sleeve 2 can be used without shaking or falling, thereby ensuring stability in using the protective sleeve; furthermore, the protective sleeve is assembled with the syringe 3 by the connection part 11 of the pipe body 1, so the protective sleeve can be applicable to most types of the syringe 3 in market, and it is not necessary to change the structural design of the syringe 3. As a result, convenience in using the protective sleeve is improved.

Thirdly, the pipe body 1 is made of transparent material and the outer sleeve 2 is provided with at least one hollow part 23 cut therethrough, so during injection operation, the injector can observe, through the hollow part 23, the injection condition of the medicinal liquid stored in the cylinder 31 of the syringe 3, to accurately inject the medicinal liquid; furthermore, the surface of the outer sleeve 2 can be marked with text message or image message of instructions for use, warning instructions or slogans, so that the protective sleeve can be applied for multiple purposes.

Fourthly, the outer sleeve 2 includes the tapered part 221 formed between the covering part 22 and the hollow body 21, so the hole diameter of the covering part 22 is gradually reduced towards the covering part 22, and the injector's finger can be prevented from contacting the tip of the needle 33 of the syringe 3 through opening of the covering part 22, to achieve objective of indeed preventing infection during the recycling process.

The present disclosure disclosed herein has been described by means of specific embodiments. However, numerous modifications, variations and enhancements can be made thereto by those skilled in the art without departing from the spirit and scope of the disclosure set forth in the claims.

What is claimed is:

1. A protective sleeve for a syringe, and the protective sleeve comprising:
a pipe body in a hollow shape and provided with a channel passing therethrough axially, and comprising a connection part formed at a side thereof and configured to combine with a syringe; and
an outer sleeve comprising a hollow body movably and extendably mounted on said pipe body, wherein said hollow body comprises a through hole passing therethrough axially and configured for insertion of said pipe body, and a covering part formed at an end thereof;
wherein before said outer sleeve is extended, a needle of said syringe is extended out of said covering part, and after said outer sleeve is extended, said needle of said syringe is accommodated inside said covering part.

2. The protective sleeve according to claim 1, wherein said pipe body is made of transparent material.

3. The protective sleeve according to claim 1, wherein said connection part of said pipe body comprises two clamping parts bent outwardly from the two opposite sides thereof to form hook shape and configured to clamp a push plate of a cylinder of said syringe.

4. The protective sleeve according to claim 1, wherein said pipe body comprises at least one position-limiting block protruded on a surface of an end thereof and spaced apart from said connection part by a distance, and at least two stop blocks protruded on a surface of other end thereof and axially arranged in interval;
wherein said outer sleeve comprises at least one hollow part cut therethrough, and a position-limiting part formed between said hollow part and an end of said outer sleeve;
wherein before said outer sleeve is extended, said position-limiting part is limited between said connection part and said position-limiting block, and after said outer sleeve is extended, said position-limiting part is limited between said at least two stop blocks arranged axially.

5. The protective sleeve according to claim 4, wherein said position-limiting block of said pipe body is provided with an arc-shaped guiding surface thereon.

6. The protective sleeve according to claim 4, wherein said at least two stop blocks of said pipe body comprise guiding inclines separately formed on outer side surfaces thereof opposite to each other, and stop surfaces formed on inner side surfaces thereof opposite to each other and configured to stop an outer surface of said position-limiting part and a sidewall of said at least one hollow part, respectively.

7. The protective sleeve according to claim 4, wherein said at least one hollow part of said outer sleeve comprises a supporting surface formed on a side surface thereof adjacent to said position-limiting part.

8. The protective sleeve according to claim 1, wherein said pipe body comprises a cut hole cut through the surface thereof.

9. The protective sleeve according to claim 1, wherein said covering part of said outer sleeve comprises a tapered part formed on a side thereof adjacent to the side of said hollow body, and a hole diameter of said covering part is gradually reduced in said tapered part.

10. The protective sleeve according to claim 1, wherein the hole diameter of said covering part of said outer sleeve is smaller than a hole diameter of said through hole.
